(19) United States
(12) Patent Application Publication
(10) Pub. No.: US 2019/0314423 A1
(43) Pub. Date: Oct. 17, 2019

(54) PLACENTAL MESENCHYMAL STEM CELLS, METHODS OF PRODUCTION, AND PLACENTAL MESENCHYMAL STEM CELL BASED THERAPEUTICS

(71) Applicant: Yuping WANG, Shreveport, LA (US)

(72) Inventor: Yuping WANG, Shreveport, LA (US)

(73) Assignee: Board of Supervisors of Louisiana State University and Agricultural and Mechanical College, Baton Rouge, LA (US)

(21) Appl. No.: 16/386,212

(22) Filed: Apr. 16, 2019

Related U.S. Application Data
(60) Provisional application No. 62/658,084, filed on Apr. 16, 2018.

(51) Int. Cl. A61K 35/50; C12N 5/073; C12N 5/079; C12N 5/0793; C12N 5/071; A61P 25/28; A61P 3/10; A61K 9/00

(52) U.S. Cl. CPC A61K 35/50; C12N 5/0605; C12N 5/0622; C12N 5/0619; A61K 9/0014; A61P 25/28; A61P 3/10; A61K 9/0019; C12N 5/0676

(57) ABSTRACT

A replicating in vitro cell culture of human placental mesenchymal stem cells comprising; cells which maintain a potential to differentiate to derivatives of multiple progenitor cell types throughout the culture. The progenitor cell types may include neuronal cells, oligodendrocytes and beta-cells. A method of treating a disease, disorder, or condition in a human comprising administering a pharmacologically effective dose of human placental mesenchymal stem cells to the human. A method for producing human placental mesenchymal stem cells.

### CROSS REFERENCE TO RELATED APPLICATIONS/PRIORITY

The present invention claims priority to U.S. Provisional Patent Application No. 62/658,084 filed Apr. 16, 2018, which is incorporated by reference into the present disclosure as if fully restated herein. Any conflict between the incorporated material and the specific teachings of this disclosure shall be resolved in favor of the latter. Likewise, any conflict between an art-understood definition of a word or phrase and a definition of the word or phrase as specifically taught in this disclosure shall be resolved in favor of the latter.

### BACKGROUND

Stem cell therapies offers tremendous promise of treatment for many diseases, including those that are currently without effective therapy. However, stem cells available in current technology have drawbacks. Bone marrow stem cells or inducible pluripotent stem cells (iPSC) from adult cells/tissue are ethically controversial, limited in quantity, and may be genetically unsuitable. Fetal stem cells are even more ethically controversial, among other issues. For the foregoing reasons, there is a pressing, but seemingly irresolvable need for developing a source of ethically uncontroversial, genetically suitable source of stem cells that are available in large quantity.

### SUMMARY

Wherefore, it is an object of the present invention to overcome the above-mentioned shortcomings and drawbacks associated with the current technology. The present invention is directed to methods and apparatuses that satisfy the above shortcomings and drawbacks. The method and apparatus comprise digested placental microvilli cultures, an elegant, efficient, and reproducible process to obtain stromal/mesenchymal stem (stem cell-like) cells from human term placenta. Placental MSCs (PMSCs) can be commercialized for research community and used for cell-based therapies for regenerations medicine. Isolation and culture/generation of mesenchymal stem cells from the human placental microvilli is a novel technique. Placenta is a medical waste and placenta-derived MSCs are not ethically restricted.

The presently claimed or disclosed invention relates to methods and replicating in vitro cell cultures of human placental mesenchymal stem cells comprising cells which maintain a potential to differentiate to derivatives of multiple progenitor cell types throughout the culture. According to a further embodiment the progenitor cell types include neuronal cells, oligodendrocytes and beta-cells. According to a further embodiment the mesenchymal stem cell have positive expression of mesenchymal stem cell markers. According to a further embodiment the mesenchymal stem cell markers are one of CD73 and CD90. According to a further embodiment the mesenchymal stem cells have negative expression of HLA-DR and CD34. According to a further embodiment the mesenchymal stem cells have positive expression of stem cell/progenitor cell markers. According to a further embodiment the stem cell/progenitor cell markers are one of Oct-4 and CD133. According to a further embodiment the mesenchymal stem cells have positive expression of neural stem/progenitor cell markers. According to a further embodiment the neural stem/progenitor cell markers are one of nestin, SOX2, and beta-tubulin III. According to a further embodiment the mesenchymal stem cells have positive expression of oligodendrocyte progenitor cell markers. According to a further embodiment the oligodendrocyte progenitor cell markers are one of O4 and Oligo2. According to a further embodiment the mesenchymal stem cells have positive expression of insulin-producing progenitor cell markers. According to a further embodiment the insulin-producing progenitor cell markers are one of c-peptide and PDX-1. According to a further embodiment the mesenchymal stem cells have positive expression of mesenchymal stem cell markers, have negative expression of HLA-DR and CD34, have positive expression of stem cell/progenitor cell markers Oct-4 or CD133, have positive expression of neural stem/progenitor cell markers, have positive expression of oligodendrocyte progenitor cell markers, and have positive expression of insulin-producing progenitor cell markers.

The presently claimed or disclosed invention further relates to therapeutics and methods of treating a disease, disorder, or condition in a human comprising administering a pharmacologically effective dose of human placental mesenchymal stem cells to the human. According to a further embodiment, the disease, disorder, or condition is one of an improperly regulated blood sugar condition, a neural degenerative disorder, and a neural disorder. According to a further embodiment, the disease, disorder, or condition is one of pre-diabetes, diabetes mellitus, Parkinson's disease and Alzheimer's disease, cerebral palsy, and multiple sclerosis. According to a further embodiment, the pharmacologically effective dose is between 750,000 and 160 million placental mesenchymal stem cells and/or their derivatives. According to a further embodiment, the administration is via one or more of intramuscular injection, intravenous injection, epidural injection, epidural catheter, retrobulbar injection, subcutaneous injection, intracardiac injection, intracystic injection, intrathecal injection, by topical application, and intralesional application.

The presently claimed or disclosed invention further relates to cells and method for producing human placental mesenchymal stem cells comprising digesting enzymatically placental villous tissue with trypsin and DNase, cultivating digested microvilli with 100% fetal bovine serum (FBS) followed by Dulbecco's Modified Eagle Medium (DMEM) supplemented with fetal bovine serum and antibiotic-antimycotic solution, growing stromal cell columns, and sub-culturing placental mesenchymal stem cells.

Various objects, features, aspects, and advantages of the present invention will become more apparent from the following detailed description of preferred embodiments of the invention, along with the accompanying drawings in which like numerals represent like components. The present invention may address one or more of the problems and deficiencies of the current technology discussed above. However, it is contemplated that the invention may prove useful in addressing other problems and deficiencies in a number of technical areas. Therefore, the claimed invention should not necessarily be construed as limited to addressing any of the particular problems or deficiencies discussed herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying drawings, which are incorporated in and constitute a part of the specification, illustrate various embodiments of the invention and together with the general description of the invention given above and the detailed description of the drawings given below, serve to explain the principles of the invention. It is to be appreciated that while the accompanying drawings are to scale, the emphasis is placed on illustrating the principles of the invention. The invention will now be described, by way of example, with reference to the accompanying drawings in which:

FIGS. 1A-1C are photomicrographs of placental microvilli in culture. Representative image of microvilli after seeding and growing cell colonies on day 3 and day 5 after seeding. FIG. 1A: Microvilli after seeding; FIG. 1B: growing cell colonies on day 3; and FIG. 1C: growing cell colonies on day 5. Bar=200 μm

FIGS. 2A-2C are two photomicrographs and four charts showing placental villous stromal cells express CD44, CD73 and CD90, markers of mesenchymal stem cells. Cells derived from placenta microvilli express mesenchymal stem cells (MSC) markers. FIG. 2A: Placental microvilous cells in culture. FIG. 2B: Positive expression of CD44 detected by immunofluorescent staining; FIG. 2C: Positive expression of CD73 and CD90 and negative expression of CD34 and HLA-DR detected by flowcytometry. CD44, CD73, and CD90 are markers of mesenchymal stem cells, CD34 is a marker of endothelial progenitor cells. HLA-DR is an MHC class II cell surface receptor and it is negatively expressed in mesenchymal stem cells. Black line: unstained control, Red line: stained cells

FIG. 3 is a set of nine photomicrographs showing placental villous stromal cells express CD133 and Oct-4. Positive expression of CD133 and Oct-4 in cells derived from placental microvilli. CD133 and Oct-4 are stem cell markers. CD133 is expressed in hematopoietic stem cells, endothelial progenitor cells, neuronal and glial stem cells, etc. Oct-4 expression is associated with an undifferentiated phenotype and considered a marker for undifferentiated cells. Oct-4 is also an important pluripotent transcription factor.

FIGS. 4A-4C are photomicrographs showing Differentiation potential of placental villous stromal cells. Alizarin Red (FIG. 4A), Alcian Blue (FIG. 4B), and Oil Red O (FIG. 4C) staining in placental villous stromal cells after differentiation. Alizarin Red is an osteogenic marker to detect calcium deposition; Alcian Blue is a chondrogenic marker to detect glycosaminoglycans; and Oil Red O is an adipogenic marker to assess lipid accumulation. A: bar=100 μm; B and C: bar=50 μm

FIG. 5 is a set of nine photomicrographs showing placental villous stromal cells express nestin, O4, and Oligo2. Expression of nestin, O4, and Oligo2 in cells derived from placenta microvillous tissue is detected by immunofluorescent staining. Nestin is a neural progenitor cell marker. O4 and Oligo2 are oligodendrocyte progenitor cell markers. Bar=50 μm

FIGS. 6A-6D are four photomicrographs showing placental villous stromal cells express PDX-1 and CD24. Expression of PDX-1 (FIG. 6A) and CD24 (FIG. 6B) in cells derived from placenta microvillous tissue detected by immunofluorescent staining. PDX-1 (FIG. 6C) and CD24 (FIG. 6D) are insulin-producing progenitor cells markers. Bar=50 μm.

### DETAILED DESCRIPTION

The present invention will be understood by reference to the following detailed description, which should be read in conjunction with the appended drawings. It is to be appreciated that the following detailed description of various embodiments is by way of example only and is not meant to limit, in any way, the scope of the present invention. In the summary above, in the following detailed description, in the claims below, and in the accompanying drawings, reference is made to particular features (including method steps) of the present invention. It is to be understood that the disclosure of the invention in this specification includes all possible combinations of such particular features, not just those explicitly described. For example, where a particular feature is disclosed in the context of a particular aspect or embodiment of the invention or a particular claim, that feature can also be used, to the extent possible, in combination with and/or in the context of other particular aspects and embodiments of the invention, and in the invention generally. The term “comprises” and grammatical equivalents thereof are used herein to mean that other components, ingredients, steps, etc. are optionally present. For example, an article “comprising” (or “which comprises”) components A, B, and C can consist of (i.e., contain only) components A, B, and C, or can contain not only components A, B, and C but also one or more other components. Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number is used herein to denote the start of a range beginning with that number (which may be a range having an upper limit or no upper limit, depending on the variable being defined). For example, “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. When, in this specification, a range is given as “(a first number) to (a second number)” or “(a first number)-(a second number),” this means a range whose lower limit is the first number and whose upper limit is the second number. For example, 25 to 100 mm means a range whose lower limit is 25 mm, and whose upper limit is 100 mm. The embodiments set forth the below represent the necessary information to enable those skilled in the art to practice the invention and illustrate the best mode of practicing the invention. In addition, the invention does not require that all the advantageous features and all the advantages need to be incorporated into every embodiment of the invention.

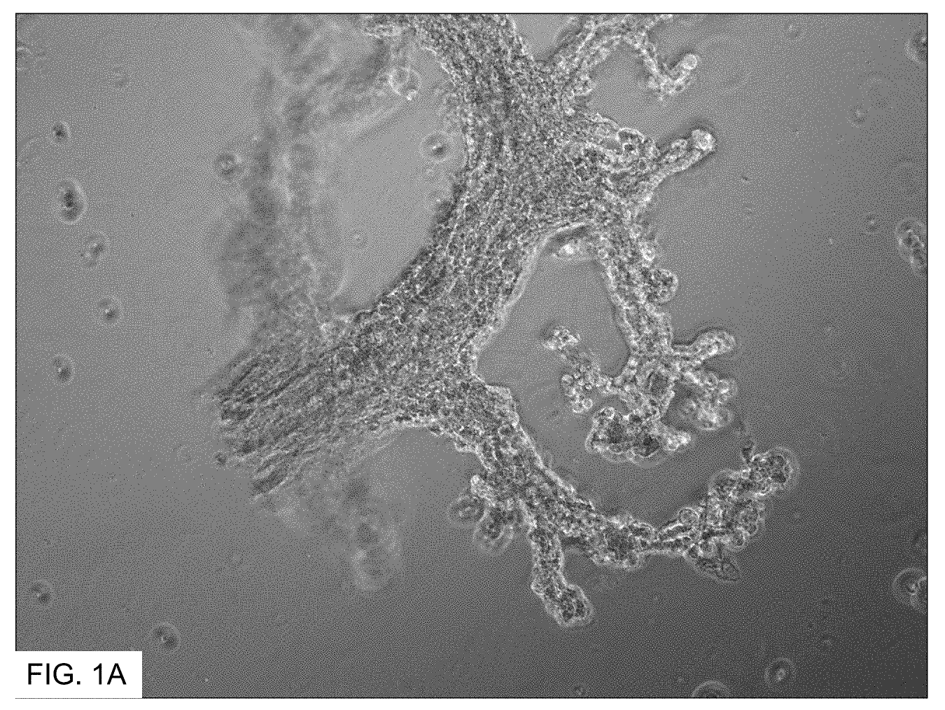

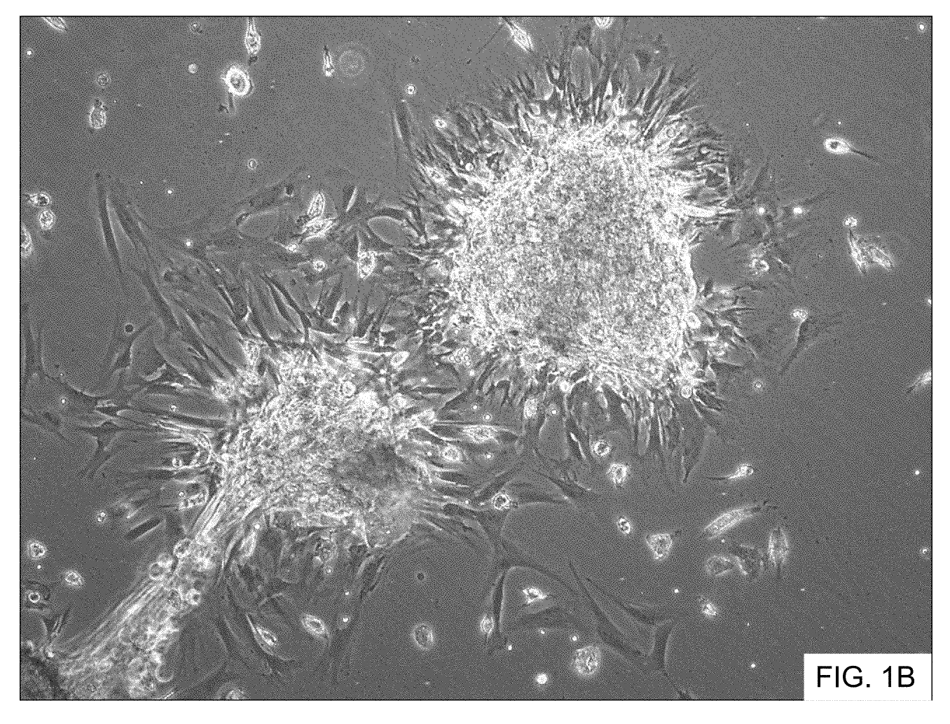

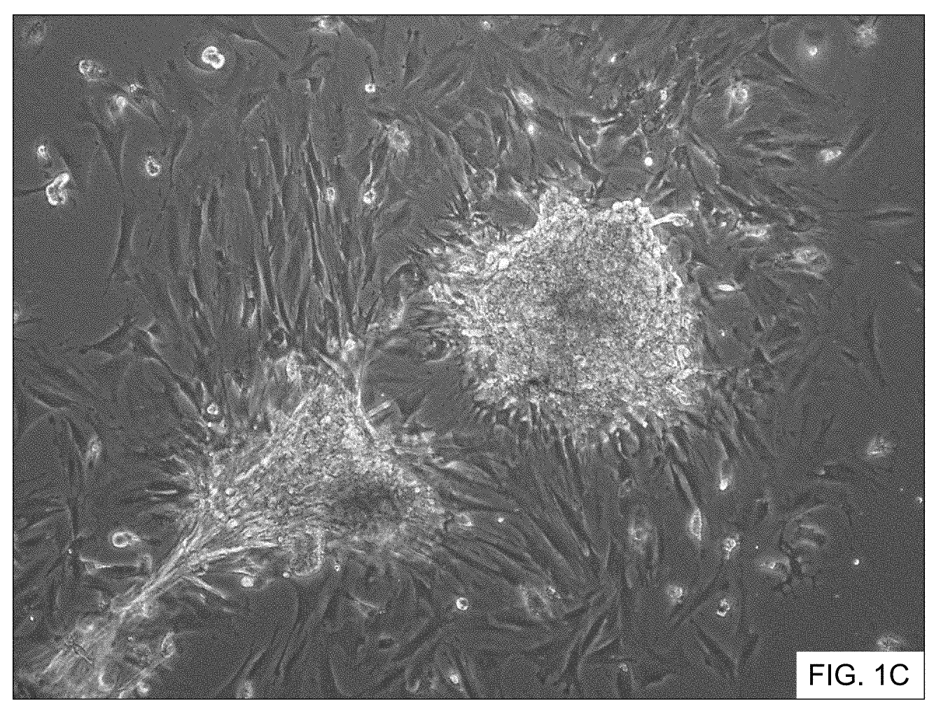

Turning now to FIGS. 1A-6D, a brief description concerning the various components of the present invention will now be briefly discussed. As can be seen in this embodiment, the presently claimed invention provides an elegant, efficient, and reproducible method for obtaining stromal/mesenchymal stem cells from human term placenta, also called placenta-derived mesenchymal stem cells (PMSCs).

Steps in the procedure include: 1) enzymatic digestion of placental villous tissue with trypsin and DNase; and 2) cultivation of digested microvilli with 100% fetal bovine serum (FBS) followed by Dulbecco's Modified Eagle Medium (DMEM) supplemented with fetal bovine serum, and antibiotic-antimycotic solution. Stromal cell columns start to grow in 2-3 days after seeding and cells can be sub cultured in 10-14 days. Cells are characterized by positive expression of stem cell/progenitor cell markers CD133 and Oct-4, mesenchymal stem cell markers CD44, CD73 and CD90, and negative expression of CD34 and HLA-DR. The placental stromal cells can be differentiated into osteocytes, chondrocytes, and adipocytes. These cells also express neural stem/progenitor cell markers nestin and SOX2, oligodendrocytes progenitor cell (OPC) markers O4 and Oligo2, and insulin-producing progenitor cell (IPC) markers PDX-1 and CD24, etc. Supporting data are shown in FIGS. 1A-6D.

FIGS. 1A-1C show representative images of microvilli after seeding and growing cell colonies on day 3 and day 5 of culture. FIG. 1A shows microvilli after seeding. FIG. 1B shows growing cell colonies on day 3. FIG. 1C shows growing cell colonies on day 5.

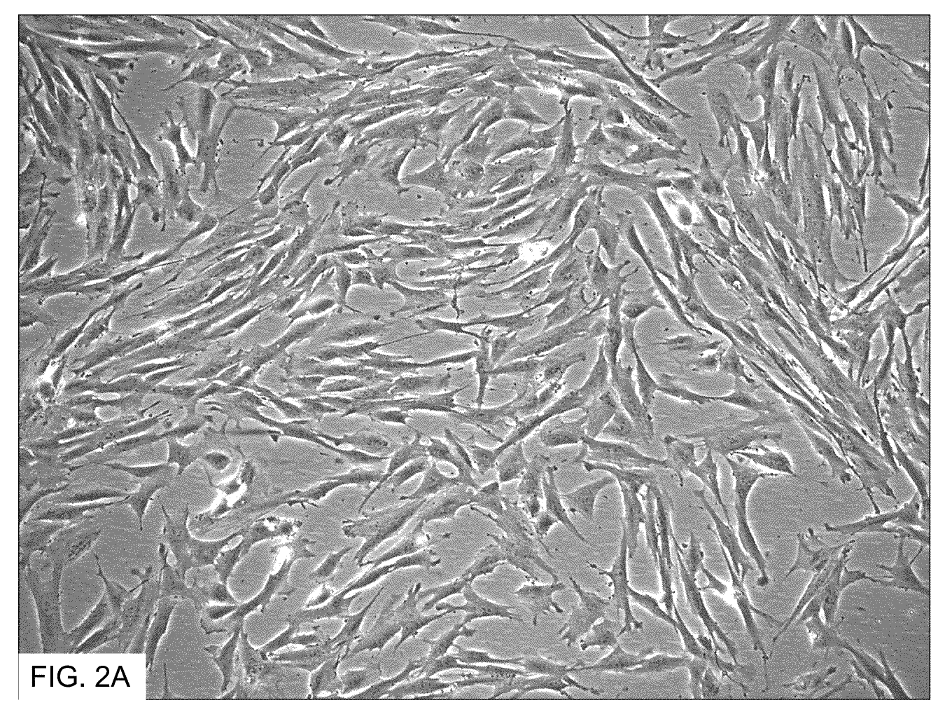

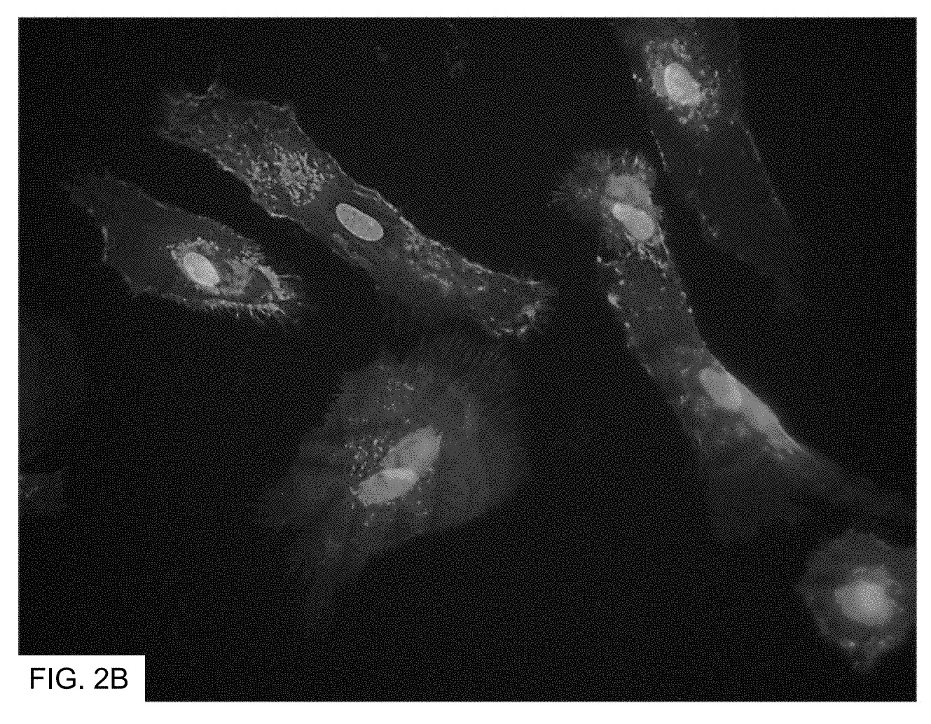

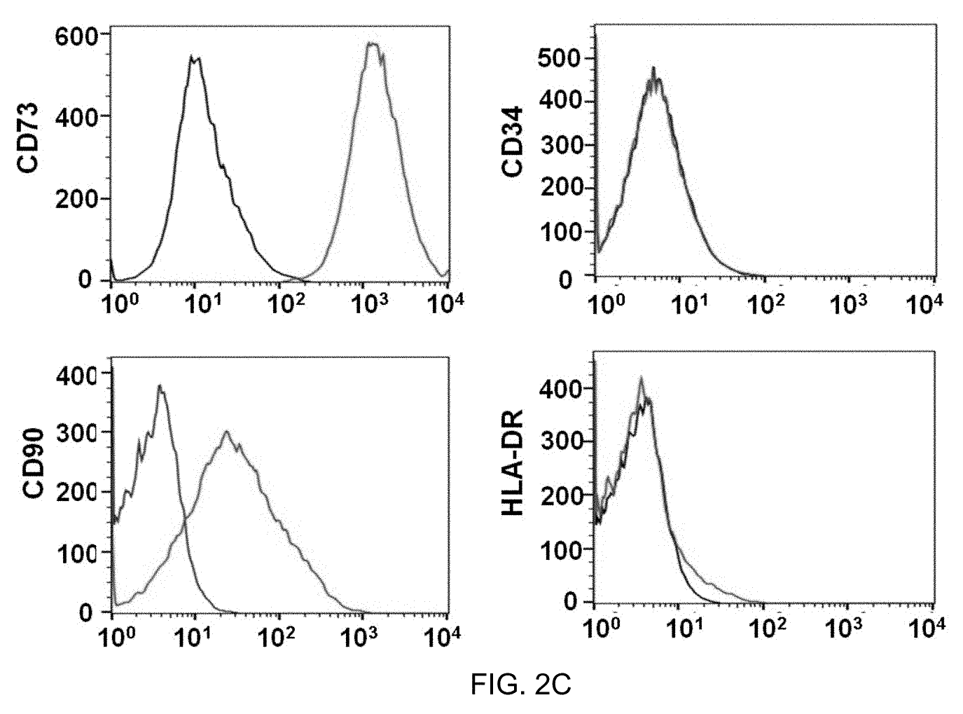

FIGS. 2A-2C show second passage of cells derived from placenta microvillous tissue with positive expression of CD44, CD73 and CD90, but not CD34 and HLA-DR. FIG. 2A shows second passage of cells derived from placenta microvillous tissue in culture. FIG. 2B shows positive expression of CD44 detected by immunofluorescent staining. FIG. 2C shows positive expression of CD73 and CD90 and negative expression of CD34 and HLA-DR detected by flowcytometry. CD44, CD73, and CD90 are markers of mesenchymal stem cells. CD34 is a marker of endothelial progenitor cells. HLA-DR is a major histocompatibility complex (MHC) class II cell surface receptor and it is negatively expressed in mesenchymal stem cells. These results indicate that cells derived from microvillous tissue are mesenchymal stem cells. Black line: unstained control, Red line: stained cells.

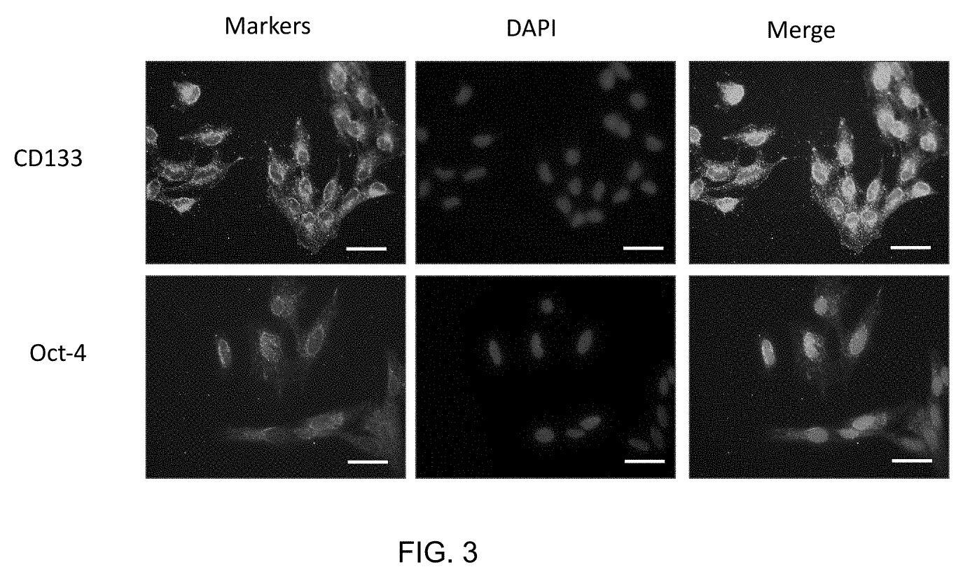

FIG. 3 shows positive expression of CD133 and Oct-4 in cells derived from placenta microvillous tissue detected by immunofluorescent staining. CD133 and Oct-4 are stem cell/progenitor cell markers. These results indicate that cells derived from microvillous tissue exert stem cell/progenitor cell properties.

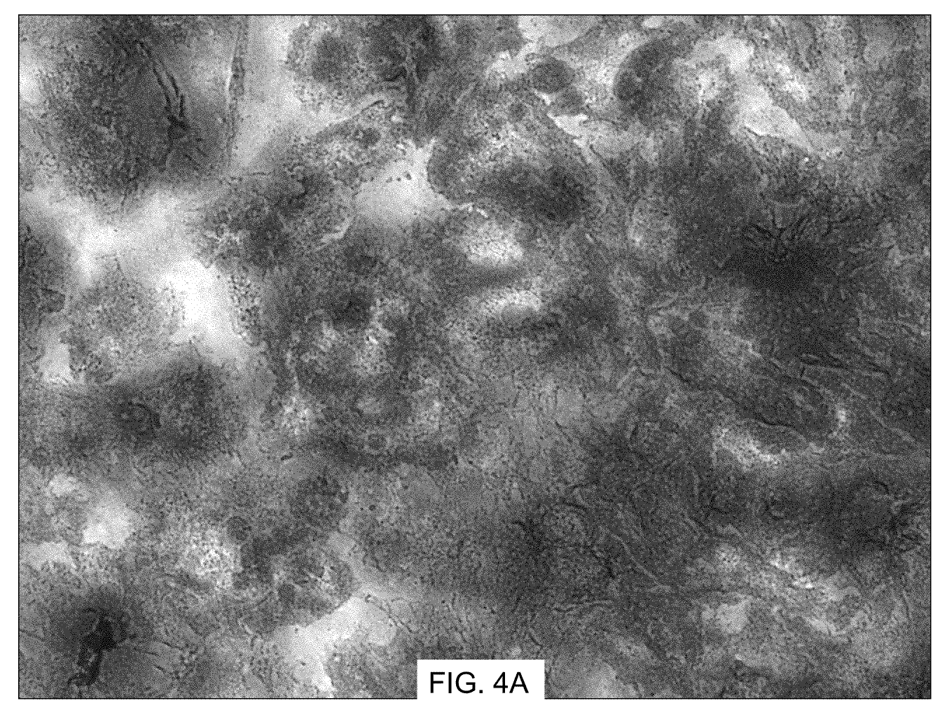

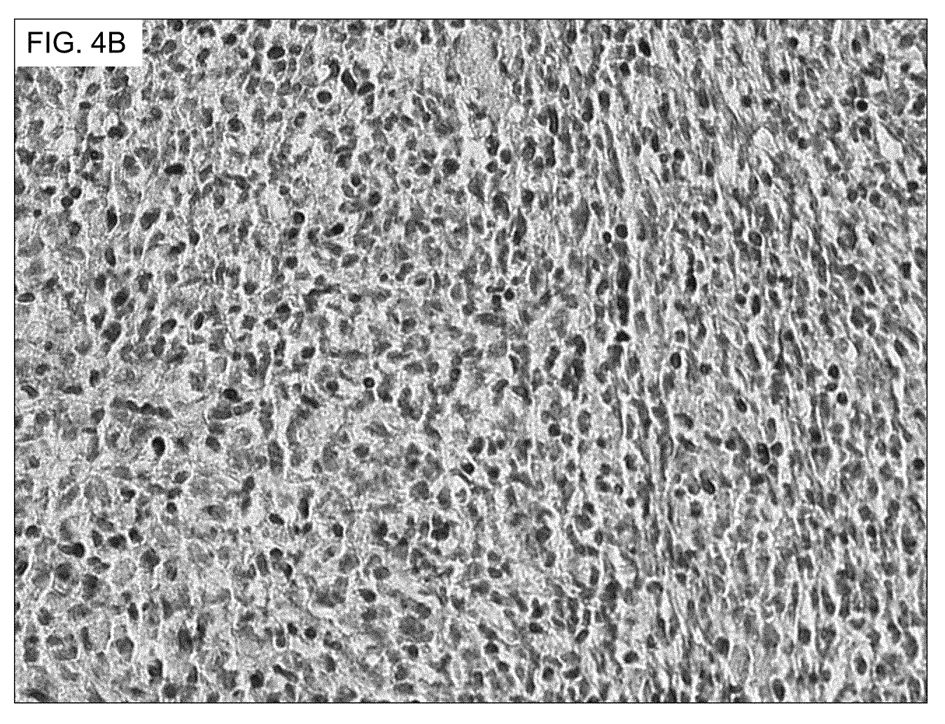

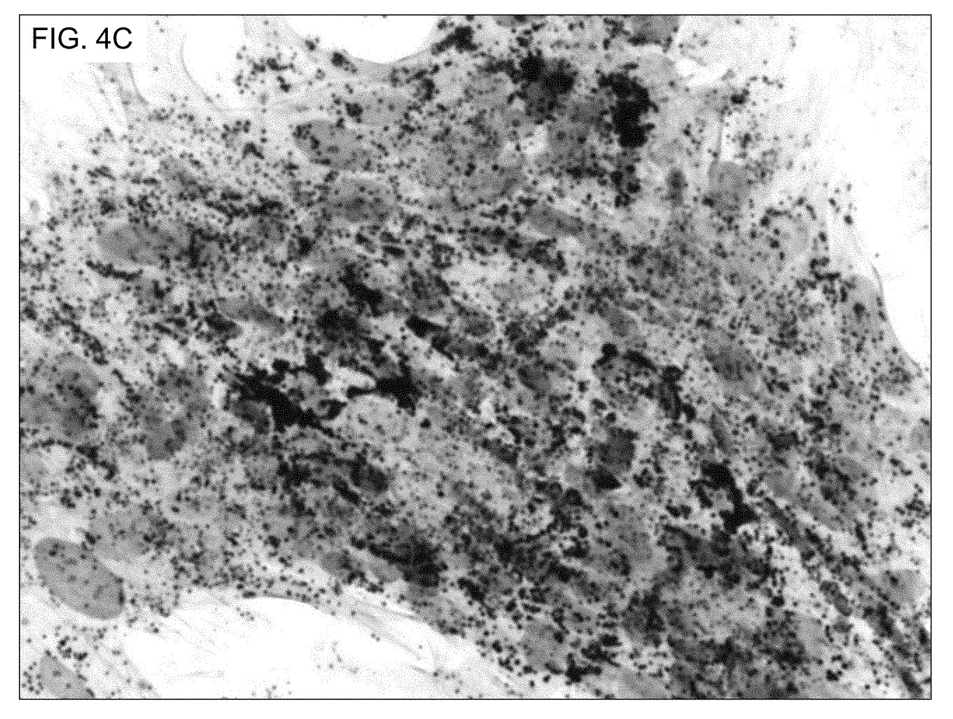

FIGS. 4A-4C show, respectively, Alizarin Red, Alcian Blue, and Oil Red O staining in cells derived from placenta microvillous tissue. Alizarin Red is an osteogenic marker to assess calcium deposition. Alcian Blue is a chondrogenic marker to detect glycosaminoglycans. Oil Red O is an adipogenic marker to assess lipid accumulation. Positive Alizarin Red, Alcian Blue, and Oil Red O staining are characteristics of mesenchymal stem cells. Therefore, the data further indicates that cells derived from placental microvillous tissue are mesenchymal stem cells and exert osteogenic, chondrogenic, and adipogenic lineage differentiation potential.

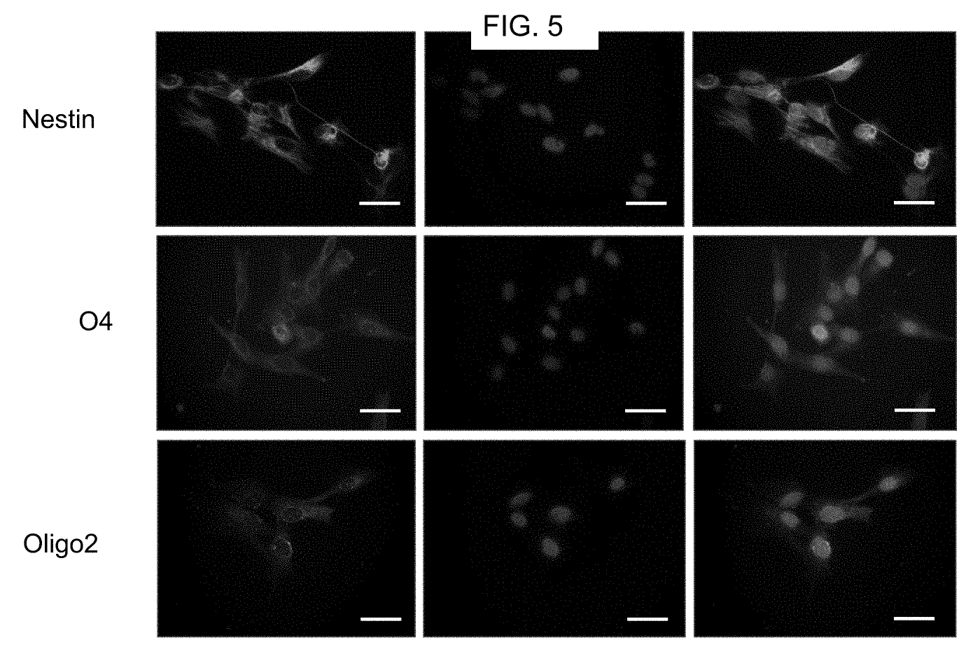

FIG. 5 shows positive expression of nestin, O4, and Oligo2 in cells derived from placenta microvillous tissue detected by immunofluorescent staining. Nestin is a neural progenitor cell marker. O4 and Oligo2 are oligodendrocyte progenitor cell markers. Positive expression of nestin, O4, and Oligo2 indicates the neuronal cell differentiation potential of placental villous mesenchymal stem cells.

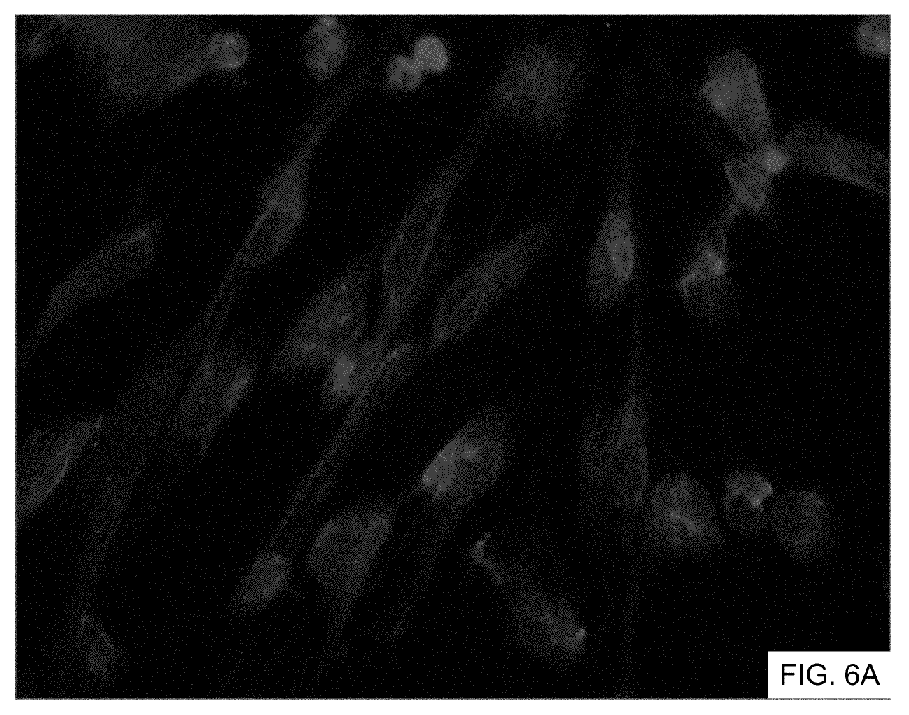

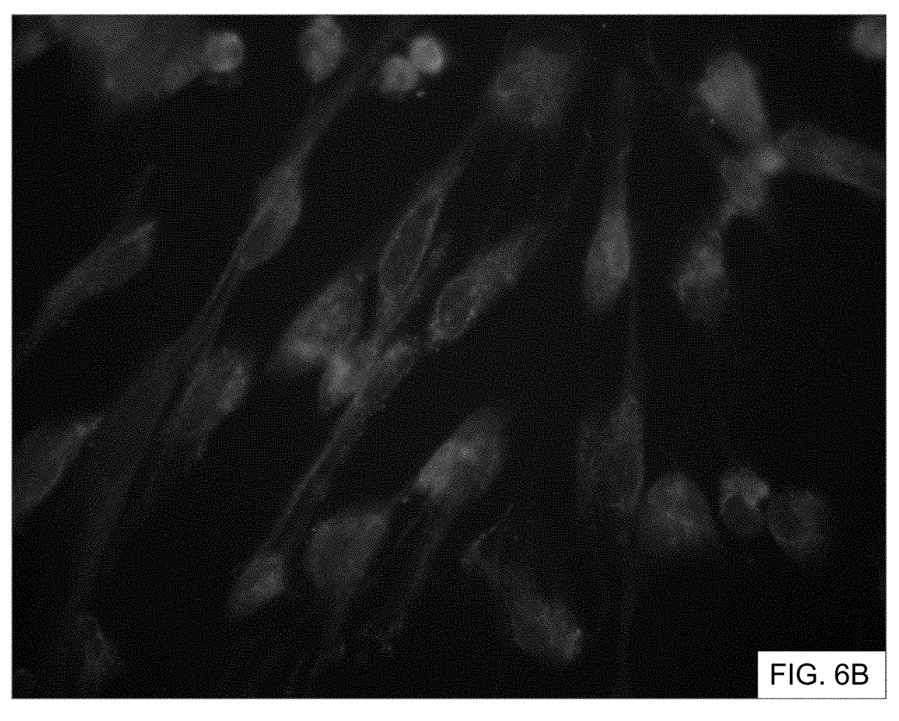

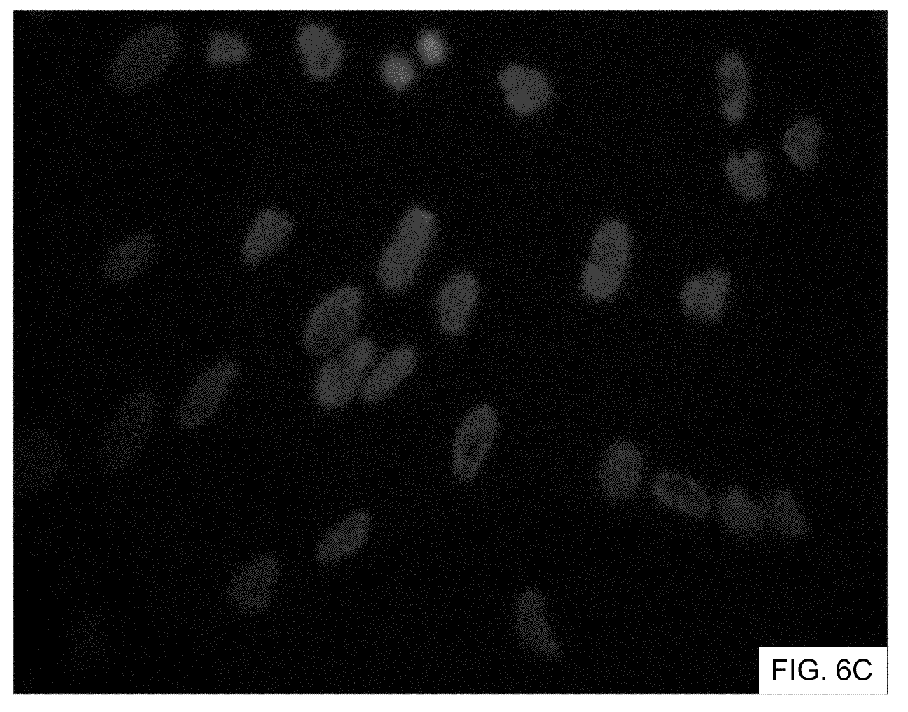

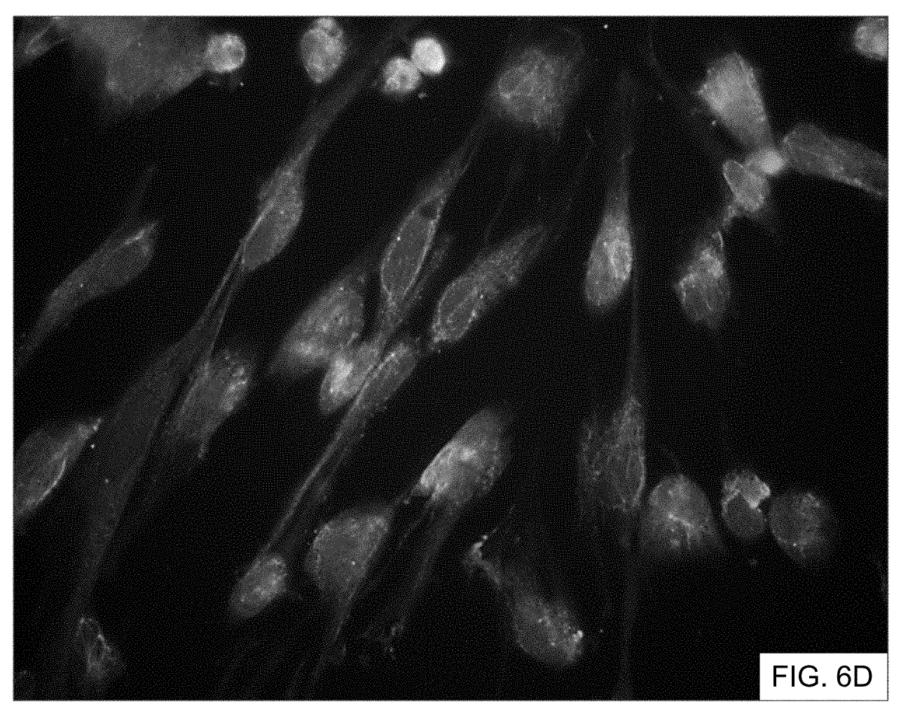

FIGS. 6A-6D show positive expression of PDX-1 and CD24 in cells derived from placenta microvillous tissue detected by immunofluorescent staining. Both PDX-1 and CD24 are insulin-producing progenitor cell markers. Positive expression of PDX-1 and CD24 indicates that placental villous mesenchymal stem cells have potential to differentiate into insulin producing cells.

The placenta is a unique “immunologically privileged organ” shared by mother and fetus during pregnancy. Human placenta represents the richest source of mesenchymal stem cells (MSCs) in human tissue. MSCs can be isolated from placenta-related tissues including amniotic fluid, cord blood, Wharton's jelly of umbilical cord, amniotic membrane, decidual basalis, and villous chorionic tissues. The placenta-derived MSCs derived from the method disclosed herein are in between embryonic and mesenchymal stem cells, sharing characteristics with both, such as non-carcinogenic status. They are pluripotent and have shown to be able to differentiate into multi-lineage cell types, including osteoblasts, chondrocytes, adipocytes, myocytes, and neural cells, for example. Compared to bone marrow stem cells or inducible pluripotent stem cells (iPSC) from adult cells/tissue, which are ethically controversial, limited in quantity, and genetically unsuitable, placenta-derived MSCs are not ethically restricted because placental tissue is considered medical waste after birth; they are pluripotent and low immunogenicity. They are lacking HLAs, making placental-derived MSCs very attractive for transplantation in allogeneic settings. The disclosed placenta-derived MSCs hold great promise for use in cell-based therapies and regeneration medicine.

Steps of the present process are 1) the establishment of an elegant, efficient, and reproducible process to obtain and culture stromal/mesenchymal stem cells from placental microvillous tissue; 2) demonstration of multi-lineage differentiation potential for osteogenesis, chondrogenesis and adipogenesis of PMSCs; 3) demonstration of pluripotent characteristics of PMSCs by positive expression of stem/progenitor cell marker CD133 and Oct-4; positive expression of neural progenitor cells nestin; positive expression of oligodendrocyte progenitor cell (OPC) markers O4 and Oligo2; and positive expression of insulin-producing progenitor cell (IPC) markers PDX-1 and CD24.

Osteogenic, chondrogenic and adipogenic properties of PMSCs are known to the inventor.

OPCs are precursors to oligodendrocytes, which can support remyelination of demyelinated axons in the central nervous system. Demyelination is the hallmark of several neurodegenerative autoimmune diseases, including multiple sclerosis (MS). Positive detection of O4 and Oligo2, markers of oligodendrocytes progenitor cells, in PMSCs indicates these cells have a potential to differentiate into oligodendrocytes and evidence the effectiveness of their use as a cell-based therapy for treatment of MS and neuronal degeneration disorders.

IPCs are precursors to insulin producing cells. Recently, insulin-producing cell therapy has been considered as a promising cell therapy for a more effective treatment of diabetes. Currently, more than 420 million people worldwide have been diagnosed with diabetes and the number continues to rise. Diabetes can cause many serous long-term complications, including cardiovascular and kidney diseases, and damage to the eyes, etc. Therefore, placenta could be an enrich source of insulin-producing progenitor cells to generate insulin producing cells as a cell-based therapy to treat diabetes.

With the pluripotent properties placental villous-derived mesenchymal stem cells would not only be a valuable tool for the research community, but also have a great potential to be used for novel cell-based therapies for regenerative and personalized medicine. These cells can also be used for drug screening.

The following procedure was used for isolation and culture of placental-derived mesenchymal stem cells (PMSCs), and is considered a preferred embodiment of the disclosed invention. Variations on this method with substitutions and alterations (in times, temperatures, chemicals, and equipment, just for example) known to those of ordinary skill in the art are also considered part of the disclosed invention.

1. Dissect placental tissue villous tissue to remove decidual basalis, chorion plate, and villous core vessels;

2. Mince dissected microvillous tissue into 1-2 mm pieces, remove blood clot and vessel pieces, and rinse villous tissue pieces intensively with phosphate buffered saline (PBS) to eliminate trapped blood and blood cells;

3. Transfer villous tissue into 50 ml conical tubes and centrifuge at 1,200 rpm for 5 min, and discard supernatant. Tissue pellet is approx. 10-12 grams;

4. Add digestion buffer (approx. 3× volume of tissue pellet), mixed well, and digest villous tissue in shaking water bath at 37° C. for 75-90 minutes. Digestion buffer 0.125% trypsin-EDTA in Dulbecco's Modified Eagle Medium (DMEM) containing 0.1 mg/mL DNase and 5 mM MgCl2 (final working concentration).

5. Discard digestion solution after centrifugation 2,000 rpm for 10 min and suspend digested tissue with cold DMEM containing 10% fetal bovine serum (FBS) and antibiotics;

6. Settle tissue (on ice) and discard supernatant (repeat this step for 3 times);

7. Centrifuge microvilli at 1,200 rpm for 5 min and discard supernatant; suspend microvilli with lysis buffer containing 155 mM NH4Cl, 10 mM KHCO3, and 0.14 mM EDTA (pH 7.2) and incubated on ice for 2-3 minutes to eliminate contaminated red blood cells;

8. Discard lysis buffer after centrifugation, 1,200 rpm for 5 min, and suspend villi with DMEM containing 10% fetal bovine serum (FBS) and antibiotics;

9. Coat 6 well/plate with 0.5 ml FBS/well;

10. Seed microvilli into FBS-coated cell culture plate (villi need to be covered by FBS) and place culture plates in a 37° C. humiliated CO2 incubator;

11. PMSC column starts to grow out in 2-4 days;

12. Add DMEM supplemented with 10% FBS and antibiotics on Day 5-6.

13. Cell columns can be removed in 1-2 weeks;

14. PMSCs should be ready to pass in 3-4 weeks.

PMSCs produced via the disclosed process may be characterized using passage 1-3 cells, MSC surface marker proteins and markers for neural progenitor cells, oligodendrocyte-progenitor cells, and insulin-producing progenitor cells, including:

A) Positive expression of mesenchymal stem cell markers such as CD73 and CD90, and negative expression of HLA-DR and CD34 by flow cytometry;

B) Positive expression of stem cell/progenitor cell markers Oct-4 or CD133 by immune-fluorescent staining;

C) Positive expression of neural stem/progenitor cell markers nestin, SOX2, and beta-tubulin III by immune-fluorescent staining;

D) Positive expression of oligodendrocyte progenitor cell markers O4 and Oligo2; and

E) Positive expression of insulin-producing progenitor cell marker c-peptide and PDX-1 by immune-fluorescent staining.

In securing the placenta selection, preferably a normal term delivery either by c-section or spontaneous vaginal delivery. Preferable to use a placenta from 37-38 weeks deliveries, with no medical and obstetrical complications, no drug and alcohol abuse, none smoker, and no fetal abnormalities.

Cell growth rate and optimal pass timing are found to be variable among the donor placentas.

The present invention provides pharmaceutical compositions comprising PMSCs and/or their derivatives, and for the use of PMSCs and their derivatives in treating a wide variety of conditions, diseases and disorders wherein the stem cells are introduced into the human body by a variety of routes of administration, topical applications or intralesional insertion.

The present invention further provides a method for treating a subject suffering from a disease, disorder or condition, including a terminal or presently incurable disease, disorder or condition comprising a schedule of administration of a therapeutically effective amount of PMSCs or their derivatives via intramuscular, intravenous, caudal, intravitreous, intrastriatal, intraparenchymal, intrathecal, epidural, retrobulbar, subcutaneous, intracardiac, intracystic, intra-articular or intrathecal injection, epidural catheter infusion, sub arachnoid block catheter infusion, intravenous infusion, via nebulizer, via spray, via intravaginal routes, via local eye and ear drops, and a schedule for administration of the PMSCs and their derivatives topically or intralesionally.

It is preferable to use PMSCs or their derivatives which are free of animal products, feeder cells, growth factors, leukemia inhibitory factor, supplementary mineral combinations, vitamin supplements, amino acid supplements, fibroblast growth factor, membrane associated steel factor, soluble steel factor and conditioned media, to avoid any chances of contamination and possibilities of negative side-effects. The PMSCs and their derivatives can be obtained through any known and approved cell culture methodology, which is feeder cell free, and free from contamination from any source and safe for human transplantation. PMSC derivatives include further differentiated cells from the human body.

PMSC Therapy

Cell-based therapy involves modifying a patient's own cells or cells from a donor to fight disease and alleviate medical conditions. In the presently disclosed invention, as the PMSCs disclosed herein are pluripotent and positively express neural-progenitor, oligodendrocyte-progenitor, and insulin-producing-progenitor cell markers, these cells have the potential to differentiate into many other cell lines, including neuronal cells, oligodendrocytes and beta-cells for therapeutic purposes.

Neural degenerative disorders treatable with PMSCs would include Parkinson's disease and Alzheimer's disease, especially in elderly people and cerebral palsy, especially in children. Neural disorders treatable with PMSCs would include oligodendrocyte injury, which leads to demyelinating disease such as multiple sclerosis (MS). Improperly regulated blood sugar conditions treatable with PMSC would include diabetes and pre-diabetes.

The present invention further provides pharmaceutical compositions for the treatment of diseases, disorders or conditions, including terminal or presently incurable diseases, disorders, or conditions comprising a therapeutically effective amount of PMSCs and/or their derivatives, wherein said PMSCs or their derivatives are preferably free of animal products, feeder cells, growth factors, leukemia inhibitory factor, supplementary mineral combinations, amino acid supplements, vitamin supplements, fibroblast growth factor, membrane associated steel factor, soluble steel factor and conditioned media, suspended in a pharmaceutically acceptable biocompatible solution or any other carrier vehicle.

The present invention also includes PMSCs and/or their derivatives free of animal products, feeder cells, growth factors, leukemia inhibitory factor, supplementary mineral combinations, amino acid supplements, vitamin supplements, fibroblast growth factor, membrane associated steel factor, soluble steel factor and conditioned media, entrapped in a biocompatible material or matrix. The biocompatible material or matrix may be selected from biopolymers, including polypeptides or proteins, polysaccharides, including fibronectin, various types of collagen, laminin, keratin, fibrin, fibrinogen, hyaluronic acid, heparin sulfate, chondroitin sulfate, agarose or gelatin.

The compositions of the present invention may be in a ready-to-use drug form in which the stem cells have adequate viability, i.e., they have a viability high enough to be useful in one or more methods of the present invention. In one embodiment, the stem cells have a viability of greater than about 40%, e.g., greater than about 50%, 60%, 70%, or 80%. The compositions may further include an antimicrobial agent, antibacterial agent, hormonal product or other pharmaceutical agent.

In order to prepare the compositions, preferably about 750,000 to about 160 million PMSCs and/or one or more of their derivatives such as different stem cell progenitors or mixtures thereof are suspended in about 0.25 ml to about 100 ml of a carrier vehicle. In one embodiment, about 750,000 to about 80 million PMSCs are suspended in about 0.25 ml to about 10 ml of the carrier vehicle. Enrichment for specific differentiated stem cell progenitor types in a population is preferable, although a proportion of undifferentiated stem cells will remain in the composition. In one embodiment, the portion of undifferentiated stem cells will be no more than about 80% of the total population of cells. In another embodiment, the portion of undifferentiated stem cells will be no more than about 40% of the total population of cells.

The invention also provides for a method for treating a subject with a disease, disorder or condition comprising administering a therapeutically effective amount of PMSCs and/or their derivatives, via intramuscular injection or intravenous injection or epidural injection or epidural catheter or retrobulbar injection or subcutaneous injection or intracardiac injection or intracystic injection or intrathecal injection or by topical application or intralesional application. In one embodiment, the disease, disorder or condition is a terminal or currently incurable disease, disorder or condition.

The invention also provides for a method for treatment of developmental, degenerative, familial and traumatic nervous system disorders, Parkinson's disease and Alzheimer's disease, cerebral palsy, multiple sclerosis, and cerebrovascular attack, comprising administration of about 750,000 to about 160 million PMSCs and/or their derivatives, via intravenous injection, subcutaneous injection, intramuscular injection, intrathecal injection, epidural catheter infusion and sub arachnoid block catheter infusion.

The invention also provides for a method for treatment of genetic disorders comprising administration of about 750,000 to about 160 million PMSCs and/or their derivatives, wherein said cells comprise neuronal stem cell progenitors, via intravenous injection, subcutaneous injection, intramuscular injection, intrathecal injection, epidural catheter infusion or sub arachnoid block catheter infusion or combinations thereof.

The invention also provides for a method for treatment of conditions associated with ageing comprising administration of about 750,000 to about 160 million PMSCs and/or their derivatives via intravenous injection, subcutaneous injection, intramuscular injection, or local application in suspension or mixed in a biocompatible carrier such as gel, ointment, matrix, paste or aerosol spray.

The invention also provides for a method for treatment of Diabetes Mellitus comprising administration of about 750,000 to about 160 million PMSCs and/or their derivatives, wherein said cells comprise insulin producing progenitor cells, via intravenous or intramuscular injection or combinations thereof.

Various exemplary routes of administration, volume, and cell numbers are provided below.

| Route of | | | | |
|---|---|---|---|---|
| administration | Volume | | Cell number | |
| intramuscular | 0.25 | ml | 750,000-1.5 | Million |
| intravenous | 0.25 | ml | 750,000-1.5 | Million |
| subcutaneous | 0.25 | ml | 750,000-1.5 | Million |
| caudal | 2 | ml | 6-16 | Million |
| epidural | 2 | ml | 6-16 | Million |
| intrathecal | 2 | ml | 6-16 | Million |
| intra-articular | 2 | ml | 6-16 | Million |
| retrobulbar | 2 | ml | 6-16 | Million |
| epidural catheter | 4-5 | ml | 12-40 | Million |
| intravenous infusion | 0.75 | ml | 2.25-4.5 | Million |
| nebulizer | 2 | ml | 6-16 | Million |
| intravaginal | 2 | ml | 6-16 | million |

The phrase “free of animal products, feeder cells, growth factors, leukemia inhibitory factor, supplementary mineral combinations, amino acid supplements, vitamin supplements, fibroblast growth factor, membrane associated steel factor, soluble steel factor and conditioned media” does not exclude the trace amounts of progestin and βhCG agonist that may be present in the pharmaceutical composition as a result of the culturing methods of the present invention. The term “animal products” refers to any non-human product.

As used herein, and as well understood in the art, “treatment” includes an approach for obtaining beneficial or desired results, such as clinical results. Beneficial or desired results can include, but are not limited to, alleviation or amelioration of one or more symptoms or conditions; diminishment of extent of disease, disorder, or condition; stabilized (i.e. not worsening) state of disease, disorder, or condition; preventing spread of disease, disorder, or condition; delay or slowing the progress of the disease, disorder, or condition; amelioration or palliation of the disease, disorder, or condition; and remission (whether partial or total), whether detectable or undetectable. “Treatment” can also mean prolonging survival as compared to expected survival if not receiving treatment. As used herein, the terms “treating” and “treatment” can also include delaying the onset of, impeding or reversing the progress of, or alleviating either the disease or condition to which the term applies, or one or more symptoms of such disease or condition.

The amount and frequency of administration of the compositions can vary depending on, for example, what is being administered, the state of the patient, and the manner of administration. In therapeutic applications, compositions can be administered to a patient suffering from a disease, disorder, or condition in an amount sufficient to relieve or least partially relieve the symptoms of the disease, disorder, or condition and its complications. The dosage is likely to depend on such variables as the type and extent of progression of the disease, disorder, or condition, the severity of the disease, disorder, or condition, the age, weight and general condition of the particular patient, the relative biological efficacy of the composition selected, formulation of the excipient, the route of administration, and the judgment of the attending clinician. Effective doses can be extrapolated from dose-response curves derived from in vitro or animal model test system. An effective dose is a dose that produces a desirable clinical outcome by, for example, improving a sign or symptom of the disease, disorder, or condition or slowing its progression

The invention illustratively disclosed herein suitably may explicitly be practiced in the absence of any element which is not specifically disclosed herein. While various embodiments of the present invention have been described in detail, it is apparent that various modifications and alterations of those embodiments will occur to and be readily apparent those skilled in the art. However, it is to be expressly understood that such modifications and alterations are within the scope and spirit of the present invention, as set forth in the appended claims. Further, the invention(s) described herein is capable of other embodiments and of being practiced or of being carried out in various other related ways. In addition, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items while only the terms “consisting of” and “consisting only of” are to be construed in the limitative sense.

Wherefore, I/We claim:

1. A replicating in vitro cell culture of human placental mesenchymal stem cells comprising;
cells which maintain a potential to differentiate to derivatives of multiple progenitor cell types throughout the culture.

2. The mesenchymal stem cells of claim 1 wherein the progenitor cell types include neuronal cells, oligodendrocytes and beta-cells.

3. The mesenchymal stem cells of claim 1 wherein the mesenchymal stem cell have positive expression of mesenchymal stem cell markers.

4. The mesenchymal stem cells of claim 3 wherein the mesenchymal stem cell markers are one of CD73 and CD90.

5. The mesenchymal stem cells of claim 1, wherein the mesenchymal stem cells have negative expression of HLA-DR and CD34.

6. The mesenchymal stem cells of claim 1, wherein the mesenchymal stem cells have positive expression of stem cell/progenitor cell markers.

7. The mesenchymal stem cells of claim 6 wherein the stem cell/progenitor cell markers are one of Oct-4 and CD133.

8. The mesenchymal stem cells of claim 1, wherein the mesenchymal stem cells have positive expression of neural stem/progenitor cell markers.

9. The mesenchymal stem cells of claim 8 wherein the neural stem/progenitor cell markers are one of nestin, SOX2, and beta-tubulin III.

10. The mesenchymal stem cells of claim 1, wherein the mesenchymal stem cells have positive expression of oligodendrocyte progenitor cell markers.

11. The mesenchymal stem cells of claim 10 wherein the oligodendrocyte progenitor cell markers are one of O4 and Oligo2.

12. The mesenchymal stem cells of claim 1, wherein the mesenchymal stem cells have positive expression of insulin-producing progenitor cell markers.

13. The mesenchymal stem cells of claim 12 wherein the insulin-producing progenitor cell markers are one of c-peptide and PDX-1.

14. The mesenchymal stem cells of claim 1 wherein the mesenchymal stem cells have positive expression of mesenchymal stem cell markers, have negative expression of HLA-DR and CD34, have positive expression of stem cell/progenitor cell markers Oct-4 or CD133, have positive expression of neural stem/progenitor cell markers, have positive expression of oligodendrocyte progenitor cell markers, and have positive expression of insulin-producing progenitor cell markers.

15. A method of treating a disease, disorder, or condition in a human comprising:
administering a pharmacologically effective dose of human placental mesenchymal stem cells to the human.

16. The method of treating a disease, disorder, or condition of claim 15, wherein the disease, disorder, or condition is one of an improperly regulated blood sugar condition, a neural degenerative disorder, and a neural disorder.

17. The method of treating a disease, disorder, or condition of claim 15, wherein the disease, disorder, or condition is one of pre-diabetes, diabetes mellitus, Parkinson's disease and Alzheimer's disease, cerebral palsy, and multiple sclerosis.

18. The method of treating a disease, disorder, or condition of claim 15, wherein the pharmacologically effective dose is between 750,000 and 160 million placental mesenchymal stem cells and/or their derivatives.

19. The method of treating a disease, disorder, or condition of claim 15 wherein the administration is via one or more of intramuscular injection, intravenous injection, epidural injection, epidural catheter, retrobulbar injection, subcutaneous injection, intracardiac injection, intracystic injection, intrathecal injection, by topical application, and intralesional application.

20. A method for producing human placental mesenchymal stem cells comprising:
digesting enzymatically placental villous tissue with trypsin and DNase;
cultivating digested microvilli with 100% fetal bovine serum (FBS) followed by Dulbecco's Modified Eagle Medium (DMEM) supplemented with fetal bovine serum and antibiotic-antimycotic solution;
growing stromal cell columns; and
sub-culturing placental mesenchymal stem cells.
